(19) United States
(12) Patent Application Publication
(10) Pub. No.: US 2025/0266136 A1
(43) Pub. Date: Aug. 21, 2025

(54) SYSTEM AND METHOD FOR DATA AUGMENTATION FOR CLINICAL NATURAL LANGUAGE PROCESSING

(71) Applicant: GE Precision Healthcare LLC, Waukesha, WI (US)

(72) Inventors: Ilanit SOBOL, Haifa (IL); Reuth GOLDSTEIN, Haifa (IL); Renato KESHET, Haifa (IL)

(21) Appl. No.: 18/443,993

(22) Filed: Feb. 16, 2024

(51) Int. Cl. G16H 10/60; G06F 40/169; G06F 40/289

(52) U.S. Cl. CPC G16H 10/60; G06F 40/169; G06F 40/289

(57) ABSTRACT

Various systems and methods are provided for generating augmented medical data. Annotated medical data including text and annotations of the text including an entity label and an assertion label may be received. A first set of words related to the assertion label may be replaced with a second set of words. A first entity in the text may be replaced with a second entity related to the entity label. Augmented medical data may be generated based on replacing the first set of words and/or replacing the first entity with the second entity. A computer executed task may be performed using the augmented medical data.

### TECHNICAL FIELD

The present disclosure relates, generally, to a system and method for data augmentation for clinical natural language processing (NLP). More specifically, the present disclosure relates to a system and method for generating augmented medical data by replacing words associated with assertion labels and/or entity labels in annotated medical data.

### BACKGROUND

Data augmentation may refer to a technique for training machine learning models using modified versions of existing data. For example, data augmentation may refer to a technique used in machine learning and computer vision to artificially increase the diversity of a training dataset by applying various transformations (e.g., rotation, cropping, flipping, etc.) to the original data. Data augmentation may help improve model generalization and performance. Clinical NLP may refer to techniques for using NLP in the medical domain, and may be used for various tasks such as information retrieval, information extraction, text classification, document classification, question answering, text summarization, etc. Clinical NLP models might require an extensive amount of training data in order to accurately perform respective tasks. Such training data might require annotation by domain experts in the form of entity labels and assertion labels. In the medical domain, a sufficient amount of appropriate training data might be difficult, or impossible, to obtain based on the expertise needed to accurately annotate the training data. Accordingly, techniques for data augmentation for clinical NLP models may improve model performance.

### SUMMARY

This summary introduces concepts that are described in more detail in the detailed description. It should not be used to identify essential features of the claimed subject matter, nor to limit the scope of the claimed subject matter.

According to an aspect of an example embodiment, a method may include receiving annotated medical data including text and annotations of the text including an entity label corresponding to a first entity in the text and an assertion label corresponding to the first entity in the text; replacing the first entity corresponding to the entity label with a second entity related to the entity label or replacing a first set of words related to the assertion label with a second set of words related to the assertion label; generating augmented medical data based on replacing the first entity corresponding to the entity label with the second entity related to the entity label or replacing the first set of words related to the assertion label with the second set of words related to the assertion label; and performing a computer executed task using the augmented medical data.

According to another aspect of an example embodiment, a method may include receiving annotated medical data including first text and annotations of the first text including an entity label corresponding to an entity in the first text and an assertion label corresponding to the entity in the first text; determining a first set of words of the first text related to the assertion label; replacing the first set of words related to the assertion label with a second set of words related to the assertion label; generating augmented medical data including second text, the entity label corresponding to the entity in the second text, and the assertion label corresponding to the entity in the second text, based on replacing the first set of words related to the assertion label with the second set of words related to the assertion label; and performing a computer executed task using the augmented medical data.

According to yet another aspect of an example embodiment, a method may include receiving annotated medical data including first text and annotations of the first text including an entity label corresponding to a first entity in the first text and an assertion label corresponding to the first entity in the first text; determining a second entity related to the entity label; replacing the first entity in the first text with the second entity related to the entity label; generating augmented medical data including second text, the entity label corresponding to the second entity in the second text, and the assertion label corresponding to the second entity in the second text, based on replacing the first entity in the second text with the second entity related to the entity label; and performing a computer executed task using the augmented medical data.

The embodiments herein provide a system and method for generating augmented medical data by replacing words associated with assertion labels and/or entity labels in medical data. Accordingly, the embodiments herein permit a large amount of training data to be generated based on a single piece of annotated medical data. In this way, the embodiments herein may quickly, efficiently, and accurately generate augmented medical data for training a clinical NLP model, and may generate clinical NLP models that are more accurate, more efficient, and/or less error-prone.

### BRIEF DESCRIPTION OF DRAWINGS

FIG. 1 is a diagram of an example system for data augmentation for clinical NLP.

FIG. 2 is a diagram of example components of a device of FIG. 1.

FIG. 3 is a flowchart of an example process for generating augmented medical data by replacing a set of words associated with an assertion label.

FIGS. 4A-4C are diagrams of an example process for generating augmented medical data by replacing a set of words associated with an assertion label.

FIG. 5 is a flowchart of an example process for generating augmented medical data by replacing an entity associated with an entity label.

FIGS. 6A-6C are diagrams of an example process for generating augmented medical data by replacing an entity associated with an entity label.

FIG. 7 is a flowchart of an example process for generating augmented medical data by replacing a set of words associated with an assertion label and by replacing an entity associated with an entity label.

FIGS. 8A and 8B are diagrams of an example process for generating augmented medical data by replacing a set of words associated with an assertion label and by replacing an entity associated with an entity label.

FIG. 9 is a diagram of an example process for training a clinical NLP model.

FIG. 10 is a diagram of an example process for training an AI model.

### DETAILED DESCRIPTION

As addressed above, a clinical NLP model may be trained to perform a particular task such as information retrieval, information extraction, text classification, document classification, question answering, text summarization, etc. To be trained to perform these tasks, the clinical NLP model might require annotated training data that includes entity labels, assertion labels, relation labels, etc. In the medical domain, such training data might be difficult, or impossible, to obtain based on the expertise needed to annotate the training data. Moreover, it might be difficult, or impossible, to obtain a sufficient amount of such training data in order to accurately train the clinical NLP model. Accordingly, clinical NLP models might be trained with an insufficient amount of training data, which can result in inaccurate, ineffective, or error-prone models.

The embodiments herein provide a system and method for generating augmented medical data by replacing words associated with assertion labels and/or entity labels in annotated medical data. Accordingly, the embodiments herein generate an expanded training data set including a large amount of training data that is developed based on a single piece of annotated medical data. The expanded training data set is developed by applying word replacement to the annotated medical data. A clinical NLP model is trained using the expanded training data set using various learning techniques. In this way, the embodiments herein may quickly, efficiently, and accurately generate augmented medical data for training and generating a robust clinical NLP model, and may generate robust clinical NLP models that are more accurate, more efficient, and/or less error-prone. Further, the embodiments herein reduce the amount of time and computational resources required for training and generating a clinical NLP model, thereby conserving processor and memory resources associated with training systems and reducing network resources. Accordingly, the embodiments herein achieve an improved technological result associated with generating and deploying clinical NLP models, and provide improvements to the technical field of clinical NLP. Moreover, the embodiments herein achieve higher performance than traditional methods that solely utilize human-annotated medical data, and are simpler by permitting a robust and expanded training data set to be generated based on reduced amount of human-annotated medical data.

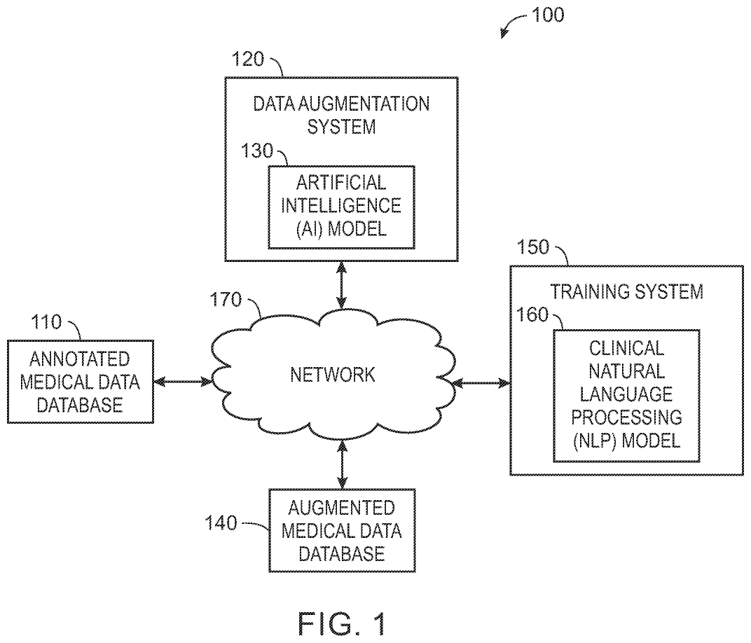

FIG. 1 is a diagram of an example system 100 for data augmentation for clinical NLP. As shown in FIG. 1, the system 100 may include an annotated medical data database 110, a data augmentation system 120, an artificial intelligence (AI) model 130, an augmented medical data database 140, a training system 150, a clinical natural language processing (NLP) model 160, and a network 170.

The annotated medical data database 110 may be configured to store annotated medical data. For example, the annotated medical data database 110 may be a hierarchical database, a network database, a relational database, a cloud database, or the like.

The data augmentation system 120 may be configured to generate augmented medical data. For example, the data augmentation system 120 may be a cloud server, a server, or the like.

The AI model 130 may be configured to generate augmented medical data. For example, the AI model 130 may be a deep neural network (DNN), a convolutional neural networks (CNN), a fully convolutional network (FCN), a recurrent neural network (RCN), a Bayesian network, a graphical probabilistic model, a K-nearest neighbor classifier, a decision forests, a maximum margin method, or the like.

The augmented medical data database 140 may be configured to store augmented medical data. For example, the augmented medical data database 140 may be a hierarchical database, a network database, a relational database, a cloud database, or the like.

The training system 150 may be configured to train the clinical NLP model 160. For example, the training system 150 may be a cloud server, a server, or the like.

The clinical NLP model 160 may be configured to perform NLP. For example, the clinical NLP model 160 may be a decision tree, a linear regression model, a neural network (e.g., a DNN, a CNN, an RNN, or the like), a transformer, a long short-term memory network, a logistic regression model, a support vector machine, or the like.

The network 170 may be configured to permit communication between the annotated medical data database 110, the data augmentation system 120, the AI model 130, the augmented medical data database 140, the training system 150, and the. For example, the network 170 may be a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of the devices of the system 100 shown in FIG. 1 are provided as an example. In practice, the system 100 may include additional devices, fewer devices, different devices, or differently arranged devices than those shown in FIG. 1. Additionally, or alternatively, a set of devices (e.g., one or more devices) of the system 100 may perform one or more functions described as being performed by another set of devices of the system 100.

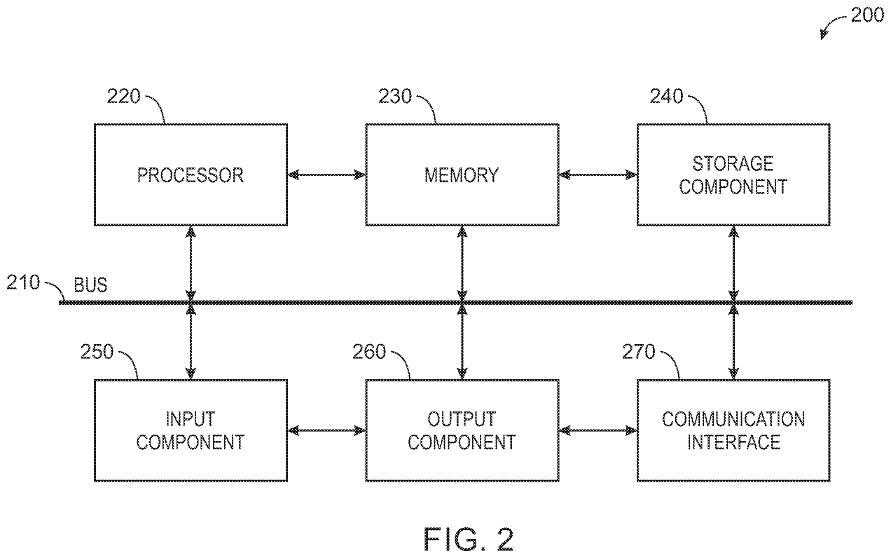

FIG. 2 is a diagram of example components of a device 200 of FIG. 1. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 220 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 220 may include one or more processors capable of being programmed to perform a function. The processor 220 may include one or more processors 220 configured to perform the operations described herein. For example, a single processor 220 may be configured to perform all of the operations described herein. Alternatively, multiple processors 220, collectively, may be configured to perform all of the operations described herein, and each of the multiple processors 220 may be configured to perform a subset of the operations descried herein. For example, a first processor 220 may perform a first subset of the operations described herein, a second processor 220 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other devices, such as via a wired connection, a wireless connection, or a combination of wired and wireless connections. The communication interface 270 may permit the device 200 to receive information from another device and/or provide information to another device. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 220 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another device via the communication interface 270. When executed, the software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

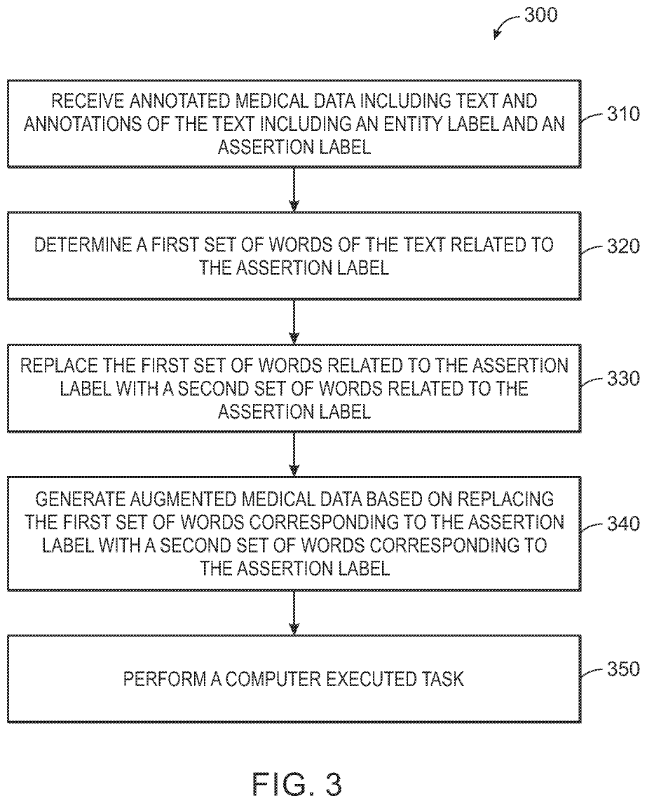

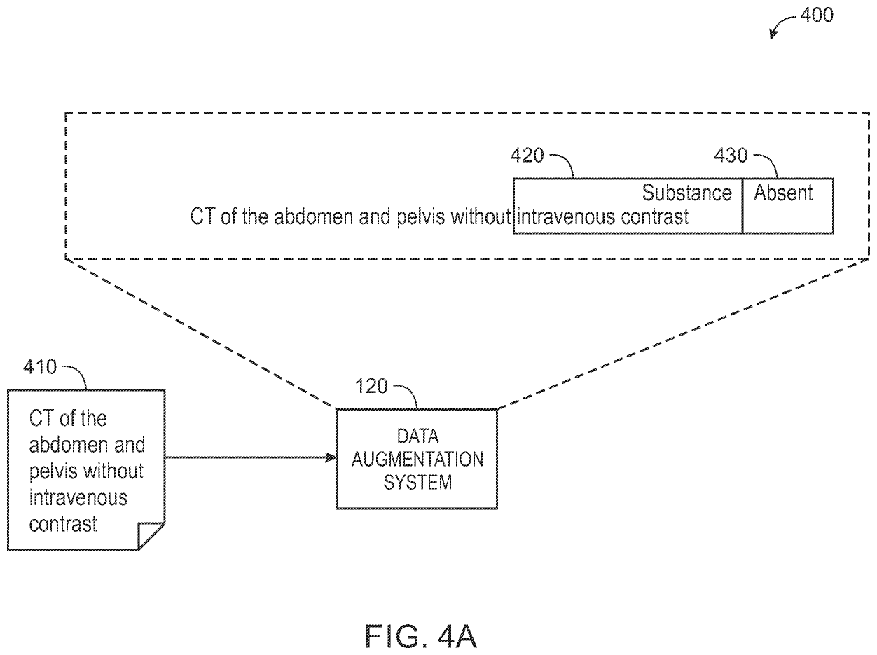

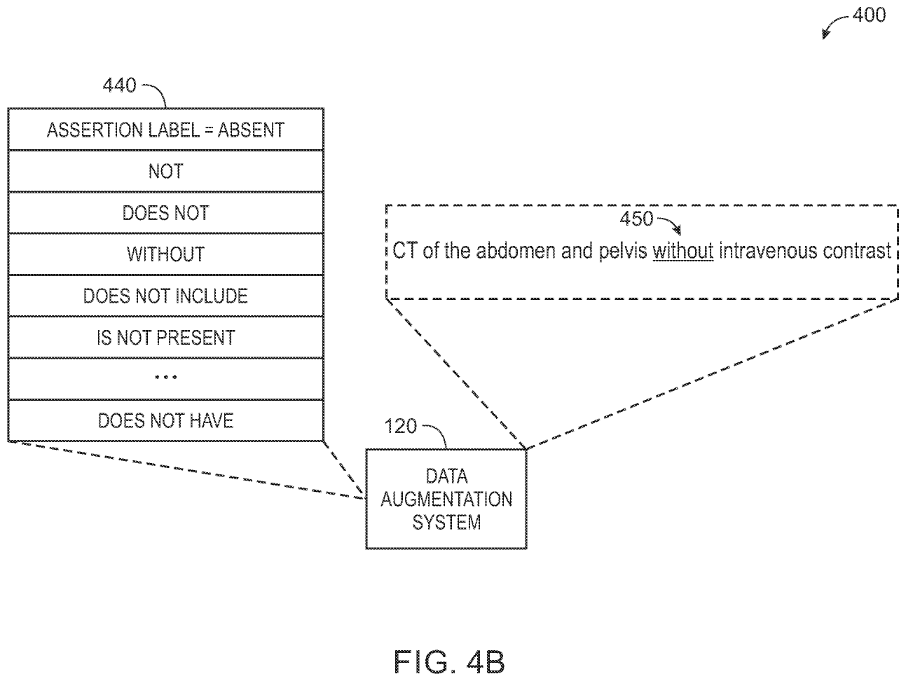

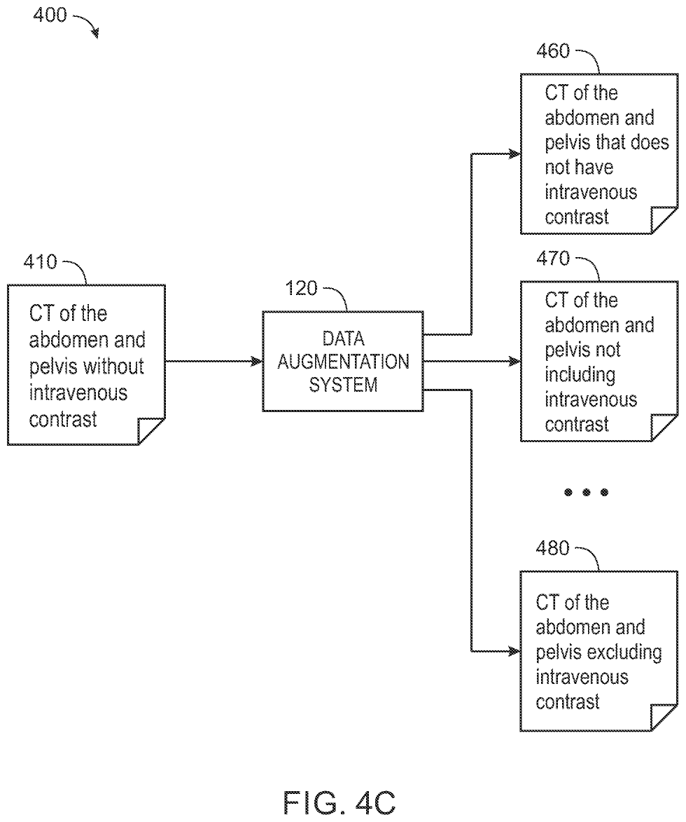

FIG. 3 is a flowchart of an example process 300 for generating augmented medical data by replacing a set of words associated with an assertion label. The data augmentation system 120 may be configured to perform the operations of the process 300. Alternatively, one or more other devices of FIG. 1 may be configured to perform one or more operations of the process 300. FIGS. 4A-4C are diagrams of an example process 400 for generating augmented medical data by replacing a set of words associated with an assertion label.

As shown in FIG. 3, the process 300 may include receiving annotated medical data including text, and annotations of the text including an entity label corresponding to an entity in the text and an assertion label corresponding to the entity in the text (operation 310).

The data augmentation system 120 may receive the annotated medical data from the annotated medical data database 110. For example, the data augmentation system 120 may receive the annotated medical data from the annotated medical data database 110 based on a request from another device, based on a user input, based on a timeframe, based on the annotated medical data being generated, based on the annotated medical data being stored in the annotated medical data database 110, or the like.

The annotated medical data may include electronic health records (EHRs), administrative data, claims data, patient data, disease data, clinical trials data, or the like. The annotated medical data may include text. For example, the annotated medical data may include text corresponding to an entity, an assertion, and a relation between the entity and the assertion. As a particular example, and as shown in FIG. 4A, the annotated medical data 410 may include the text of “CT of the abdomen and pelvis without intravenous contrast.”

The annotated medical data may include annotations of the text. The annotated medical data may include annotations provided by a domain expert, an AI model, or the like. According to an embodiment, the annotations of the annotated medical data may include an entity label. The entity label may be correspond to an entity in the text, and may correspond to a class of entities. For example, the entity label may be “patient,” “procedure,” “body part,” “drug,” “disease,” “location,” “substance,” or the like. The entity may be a specific patient, a specific procedure, a specific body part, a specific drug, a specific disease, a specific location, or the like. For example, the entity label may be “body part,” and the entity may be a specific body part such as “brain,” “liver,” “heart,” or the like. As another example, the entity label may be “disease,” and the entity may be a specific disease such as “cancer,” “diabetes,” “chronic respiratory disease,” or the like. According to an embodiment, the entity label may be a named entity recognition (NER) label.

According to another embodiment, the annotations of the annotated medical data may include an assertion label. The assertion label may be a label corresponding to the entity in the text, and may correspond to a class of assertions. For example, the assertion label may be “present,” “absent,” “possible,” “hypothetical,” “present,” “current,” “past,” or the like. As a particular example, and as shown in FIG. 4A, the annotated medical data 410 may include an entity label 420 of “substance” corresponding to the entity of “intravenous contrast” in the annotated medical data 410. Further, as shown in FIG. 4A, the annotated medical data 410 may include an assertion label 430 of “absent” corresponding to the entity of “intravenous contrast.”

As further shown in FIG. 3, the process 300 may include determining a first set of words of the text related to the assertion label (operation 320).

The first set of words of the text related to the assertion label may include one or more words related to the assertion label. For example, if the assertion label is “present,” then the first set of words may include “includes,” “is present,” “has,” “with,” “occurs,” or the like. As another example, if the assertion label is “absent,” then the first set of words may include “does not include,” “is not present,” “does not have,” “without,” “does not occur,” or the like. The annotations of the annotated medical data might not include the first set of words. Put another way, the first set of words are not annotated.

According to an embodiment, the data augmentation system 120 may determine the first set of words of the text related to the assertion label using a text analysis technique. For example, the data augmentation system 120 may determine the first set of words using a regular expression technique, string searching technique, a string manipulation technique, a sorting technique, a parsing technique, a mining technique, or the like. As a particular example, the data augmentation system 120 may use a regular expression to determine the first set of words of the text related to the assertion label. The data augmentation system 120 may determine the assertion label, and determine a particular regular expression to use based on the assertion label. For example, if the assertion label is “present,” then the data augmentation system 120 may use a regular expression corresponding to the assertion label of “present.”

According to an embodiment, the data augmentation system 120 may determine the first set of words of the text related to the assertion label using the AI model 130. For example, the data augmentation system 120 may input the text of the annotated medical data into the AI model 130, and determine the first set of words based on an output of the AI model 130. In this case, the AI model 130 may be trained to receive the text of the annotated medical data, receive the assertion label, determine the first set of words related to the assertion label, and output the first set of words related to the assertion label.

According to an embodiment, the data augmentation system 120 may determine the first set of words of the text related to the assertion label using mapping information that maps an assertion label and words related to the assertion label. For example, the data augmentation system 120 may compare the words of the mapping information with words of the text of the annotated medical data, and determine the first set of words based on comparing the words of the mapping information with the words of the text. As a particular example, and as shown in FIG. 4B, the data augmentation system 120 may receive mapping information 440 that maps the assertion label 430 of “absent” to words such as “not,” “does not,” “without,” “does not include,” “is not present,” “does not have,” or the like. The data augmentation system 120 may determine the first set of words 450 of “without” corresponding to the assertion label 430 of “absent” based on the mapping information 440.

As further shown in FIG. 3, the process 300 may include replacing the first set of words related to the assertion label with a second set of words related to the assertion label (operation 330).

The second set of words related to the assertion label may include one or more words related to the assertion label. For example, if the assertion label is “present,” then the second set of words may include “includes,” “is present,” “has,” “with,” “occurs,” or the like. As another example, if the assertion label is “absent,” then the second set of words may include “does not include,” “is not present,” “does not have,” “without,” “does not occur,” or the like. In this way, the second set of words may be contextually similar to the first set of words. However, the second set of words might be different than the first set of words. For example, if the assertion label is “present” and the first set of words is “includes,” then the second set of words may be “is present.” As another example, if the assertion label is “absent” and the first set of words is “does not include,” then the second set of words may be “is not present.”

According to an embodiment, the data augmentation system 120 may determine the second set of words based on the mapping information that maps the assertion label and words related to the assertion label. For example, the data augmentation system 120 may determine the second set of words based on the mapping information by selecting the second set of words from the mapping information.

According to an embodiment, the data augmentation system 120 may determine the second set of words using the AI model 130. For example, the data augmentation system 120 may input the first set of words and/or the assertion label into the AI model 130, and determine the second set of words based on an output of the AI model 130. In this case, the AI model 130 may be trained to receive the first set of words and/or the assertion label, determine the second set of words, and output the second set of words.

The data augmentation system 120 may replace the first set of words with the second set of words. For example, the data augmentation system 120 may replace the first set of words with the second set of words to generate augmented medical data, as described below.

As further shown in FIG. 3, the process 300 may include generating augmented medical data based on replacing the first set of words related to the assertion label with the second set of words related to the assertion label (operation 340).

The augmented medical data may be annotated medical data including the second set of words instead of the first set of words. As a particular example, and referring to FIG. 4C, the data augmentation system 120 may replace the first set of words “without” in the annotated medical data 410 with the second set of words “that does not have,” and generate augmented medical data 460 including the second set of words. As another particular example, and referring to FIG. 4C, the data augmentation system 120 may replace the first set of words “without” in the annotated medical data 410 with the second set of words “not including,” and generate the augmented medical data 470 including the second set of words. As yet another particular example, and referring to FIG. 4C, the data augmentation system 120 may replace the first set of words “without” in the annotated medical data 410 with the second set of words of “excluding,” and generate augmented medical data 480 including the second set of words. The augmented medical data may include the entity label and the assertion label from the annotated medical data. In this way, the data augmentation system 120 may generate n pieces of augmented medical data based on a single piece of input annotated medical data by replacing the first set of words in the medical data with n second sets of words.

The data augmentation system 120 may generate the augmented medical data, and store the augmented medical data in the augmented medical data database 140. In this way, the data augmentation system 120 may receive annotated medical data, generate an augmented medical data set including n variations of the annotated medical data by replacing words corresponding to an assertion label of the annotated medical data with other words corresponding to the assertion label, and store the augmented medical data set for subsequent training of the clinical NLP model 160. Accordingly, the data augmentation system 120 may quickly, efficiently, and accurately generate augmented medical data for training the clinical NLP model 160.

As further shown in FIG. 3, the process 300 may include performing a computer executed task using the augmented medical data (operation 350). For example, the computer executed task may include causing the clinical NLP model 160 to be trained, or training the clinical NLP model 160. As another example, the computer executed task may include causing the clinical NLP model 160 to be updated, or updating the clinical NLP model 160. As another example, the computer executed task may include transmitting the augmented medical data to another device. As another example, the computer executed task may include storing the augmented medical data in the augmented medical database 140. As another example, the computer executed task may include generating a user interface that displays the augmented medical data, and/or providing the user interface to another device.

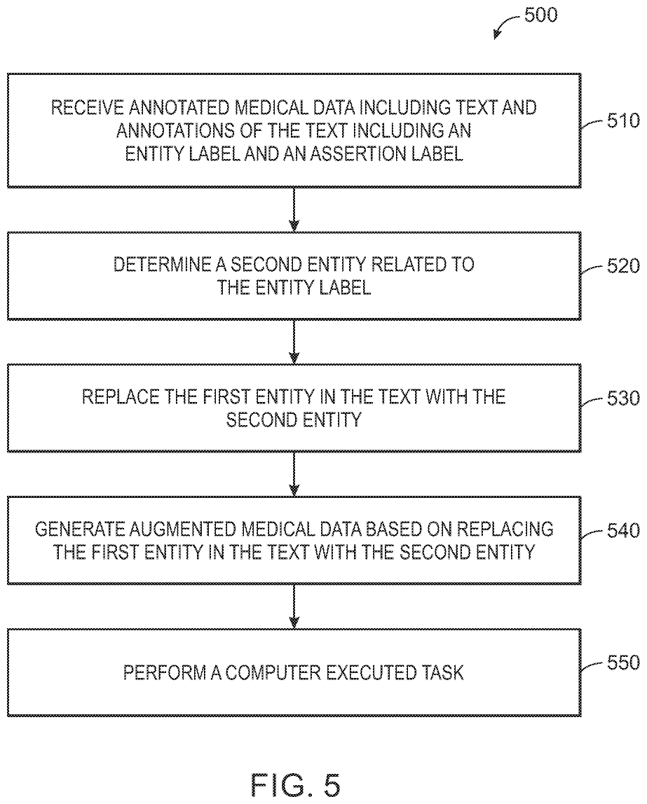

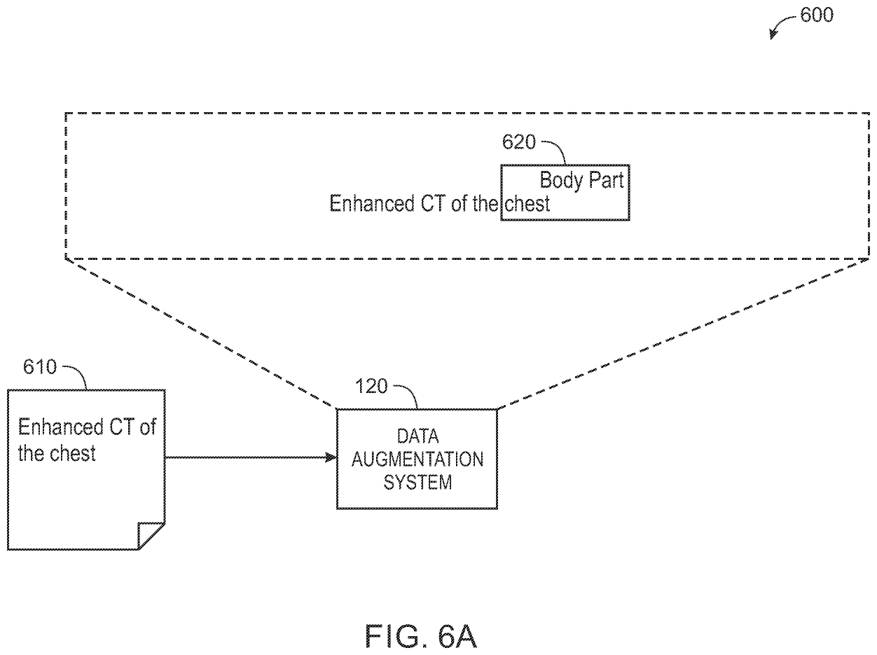

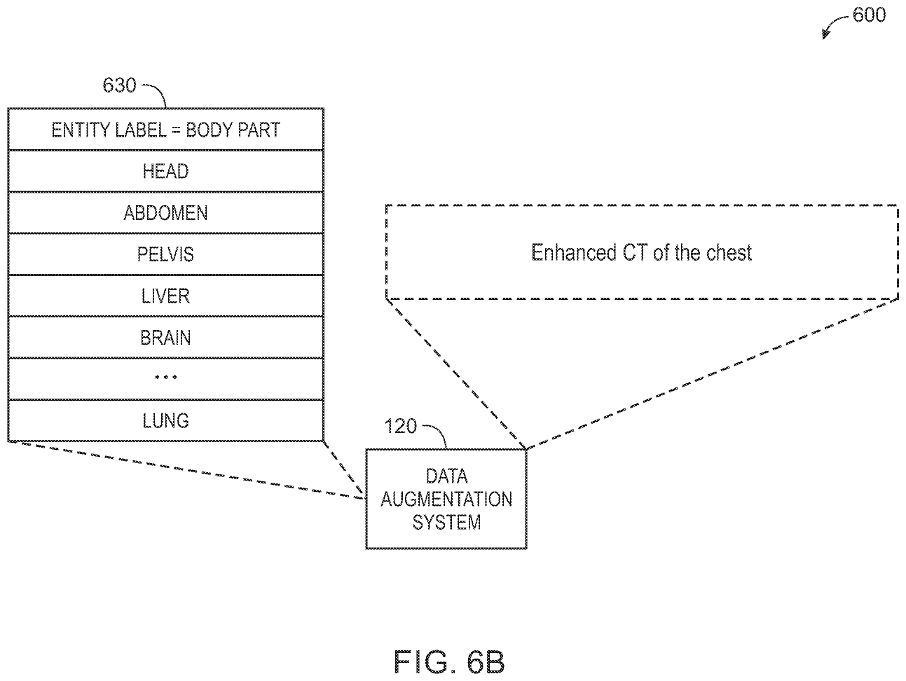

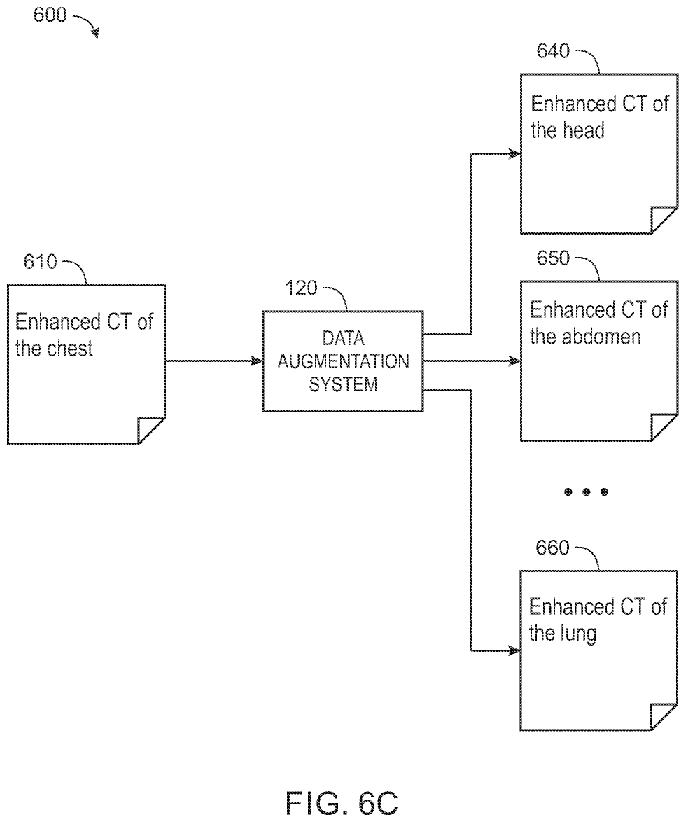

FIG. 5 is a flowchart of an example process for generating augmented medical data by replacing an entity associated with an entity label. The data augmentation system 120 may be configured to perform the operations of the process 500. Alternatively, one or more other devices of FIG. 1 may be configured to perform one or more operations of the process 500. FIGS. 6A-6C are diagrams of an example process for generating augmented medical data by replacing an entity associated with an entity label.

As shown in FIG. 5, the process 500 may include receiving annotated medical data including text, and annotations of the text including an entity label corresponding to a first entity in the text and an assertion label corresponding to the first entity in the text (operation 510).

The data augmentation system 120 may receive the annotated medical data from the annotated medical data database 110. For example, the data augmentation system 120 may receive the annotated medical data from the annotated medical data database 110 based on a request from another device, based on a user input, based on a timeframe, based on the annotated medical data being generated, based on the annotated medical data being stored in the annotated medical data database 110, or the like.

The annotated medical data may include EHRs, administrative data, claims data, patient data, disease data, clinical trials data, or the like. The annotated medical data may include text. For example, the annotated medical data may include text corresponding to a first entity, an assertion, and a relation between the first entity and the assertion. As a particular example, and as shown in FIG. 6A, the annotated medical data 610 may include the text of “Enhanced CT of the chest.”

The annotated medical data may include annotations of the text. The annotated medical data may include annotations provided by a domain expert, an AI model, or the like. According to an embodiment, the annotations of the annotated medical data may include an entity label. The entity label may be correspond to the first entity in the text, and may correspond to a class of entities. For example, the entity label may be “patient,” “procedure,” “body part,” “drug,” “disease,” “location,” “substance,” or the like. The first entity may be a specific patient, a specific procedure, a specific body part, a specific drug, a specific disease, a specific location, or the like. For example, the entity label may be “body part,” and the first entity may be a specific body part such as “brain,” “liver,” “heart,” or the like. As another example, the entity label may be “disease,” and the first entity may be a specific disease such as “cancer,” “diabetes,” “chronic respiratory disease,” or the like. According to an embodiment, the entity label may be an NER label.

According to another embodiment, the annotations of the annotated medical data may include an assertion label. The assertion label may be a label corresponding to the entity in the text, and may correspond to a class of assertions. For example, the assertion label may be “present,” “absent,” “possible,” “hypothetical,” “present,” “current,” “past,” or the like. As a particular example, and as shown in FIG. 6A, the annotated medical data 610 may include the entity label 620 of “body part” corresponding to the first entity of “chest.”

As further shown in FIG. 5, the process 500 may include determining a second entity related to the entity label (operation 520).

The second entity related to the entity label may be another entity that belongs to a same class of entities as the first entity. For example, if the entity label is “body part” and the first entity is “brain,” then the second entity may be another body part such as “liver.” As another example, if the entity label is “disease” and the first entity is “cancer,” then the second entity may be another disease such as “diabetes.”

According to an embodiment, the data augmentation system 120 may determine the second entity related to the entity label based on mapping information that maps entities with an entity label. For example, the mapping information may map specific body parts (e.g., “brain,” “liver,” “heart,” or the like) with an entity label of “body part.” As another example, the mapping information may map specific diseases (e.g., “cancer,” “diabetes,” “chronic respiratory disease,” or the like) with the entity label of “disease.” The data augmentation system 120 may determine the mapping information based on the entity label included in the annotated medical data, and determine a second entity from the mapping information. As a particular example, and referring to FIG. 6B, the data augmentation system 120 may determine mapping information 630 based on the entity label of the first entity, and determine the second entity based on the mapping information. As shown, the mapping information 630 maps various entities (e.g., “head,” “abdomen,” “pelvis,” etc.) with the entity label 620 of “body part.”

According to an embodiment, the data augmentation system 120 may generate the mapping information based on annotated medical data stored in the annotated medical data database 110. The annotated medical data may include a large number of associations between entities and entity labels. Accordingly, the data augmentation system 120 may generate the mapping information based on the associations between the entities and the entity labels, and use the mapping information to determine the second entity. According to other embodiments, the mapping information may be manually generated by domain experts, provided by a client hospital, generated based on medical repositories (e.g., medical terminologies, ontologies, encyclopedias, etc.), prompting large language models, or the like.

According to an embodiment, the data augmentation system 120 may determine the second entity based on a frequency of the second entity in the annotated medical data stored in the annotated medical data database 110. The mapping information may identify a frequency of an entity in a set of annotated medical data stored by the annotated medical data database 110. The mapping information may be generated based on the annotated medical data stored in the annotated medical data database 110. Accordingly, an entity that occurs in more pieces of annotated medical data in the annotated medical data database 110 may include a greater frequency than as compared to an entity that occurs in less pieces of annotated medical data in the annotated medical data database 110. The data augmentation system 120 may determine the second entity that includes the greatest frequency, a frequency greater than a threshold frequency, or the like. In this way, the data augmentation system 120 may improve the accuracy of the augmented medical data by using second entities that appear more frequently, that do not include typographical errors, or the like. Further, in this way, the data augmentation system may account for annotation inaccuracies in annotated medical data, thereby increasing the validity of the augmented medical data.

According to an embodiment, the data augmentation system 120 may determine the second entity using the AI model 130. For example, the data augmentation system 120 may input the first entity and/or the entity label into the AI model 130, and determine the second entity based on an output of the AI model 130. In this case, the AI model 130 may be trained to receive the first set of words and/or the assertion label, determine the second set of words, and output the second set of words.

According to an embodiment, the data augmentation system 120 may determine the second entity based on the entity label corresponding to the first entity in the text and the assertion label corresponding to the first entity in the text. For example, the data augmentation system 120 may determine annotated medical data including the same entity label and the same assertion label as included in the received annotated medical data, and determine a second entity included in the annotated medical data. As a particular example, if the received annotated medical data for which augmented medical data is to be generated includes an entity label of “disease” and an assertion label of “present” corresponding to a first entity of “cancer,” then the data augmentation system 120 may determine annotated medical data that includes the entity label of “disease” and the assertion label of “present,” and determine the second entity using the annotated medical data.

As further shown in FIG. 5, the process 500 may include replacing the first entity in the text with the second entity (operation 530).

The data augmentation system 120 may replace the first entity in the text of the annotated medical data with the second entity. For example, the data augmentation system 120 may replace the first entity with the second entity to generate augmented medical data, as described below.

As further shown in FIG. 5, the process 500 may include generating augmented medical data based on replacing the first entity in the text with the second entity (operation 540).

The augmented medical data may be annotated medical data including the second entity instead of the first entity. As a particular example, and referring to FIG. 6C, the data augmentation system 120 may replace the first entity of “chest” in the annotated medical data 610 with the second entity of “head,” and generate augmented medical data 640 including the second entity. As another particular example, and referring to FIG. 6C, the data augmentation system 120 may replace the first entity “chest” in the annotated medical data 640 with the second entity of “abdomen,” and generate the augmented medical data 650 including the second entity. As yet another particular example, and referring to FIG. 6C, the data augmentation system 120 may replace the first entity “chest” in the annotated medical data 610 with the second entity of “lung,” and generate augmented medical data 660 including the second entity. The augmented medical data may include the entity label from the annotated medical data. In this way, the data augmentation system 120 may generate n pieces of augmented medical data based on a single piece of input annotated medical data by replacing the first entity in the annotated medical data with n second entities.

The data augmentation system 120 may generate the augmented medical data, and store the augmented medical data in the augmented medical data database 140. In this way, the data augmentation system 120 may receive annotated medical data, generate an augmented medical data set including n variations of the annotated medical data by replacing words corresponding to entities of the annotated medical data with other words corresponding to the entities, and store the augmented medical data set for subsequent training of the clinical NLP model 160. Accordingly, the data augmentation system 120 may quickly, efficiently, and accurately generate augmented medical data for training the clinical NLP model 160.

As further shown in FIG. 5, the process 500 may include performing a computer executed task using the augmented medical data (operation 550). For example, the computer executed task may include causing the clinical NLP model 160 to be trained, or training the clinical NLP model 160. As another example, the computer executed task may include causing the clinical NLP model 160 to be updated, or updating the clinical NLP model 160. As another example, the computer executed task may include transmitting the augmented medical data to another device. As another example, the computer executed task may include storing the augmented medical data in the augmented medical database 140. As another example, the computer executed task may include generating a user interface that displays the augmented medical data, and/or providing the user interface to another device.

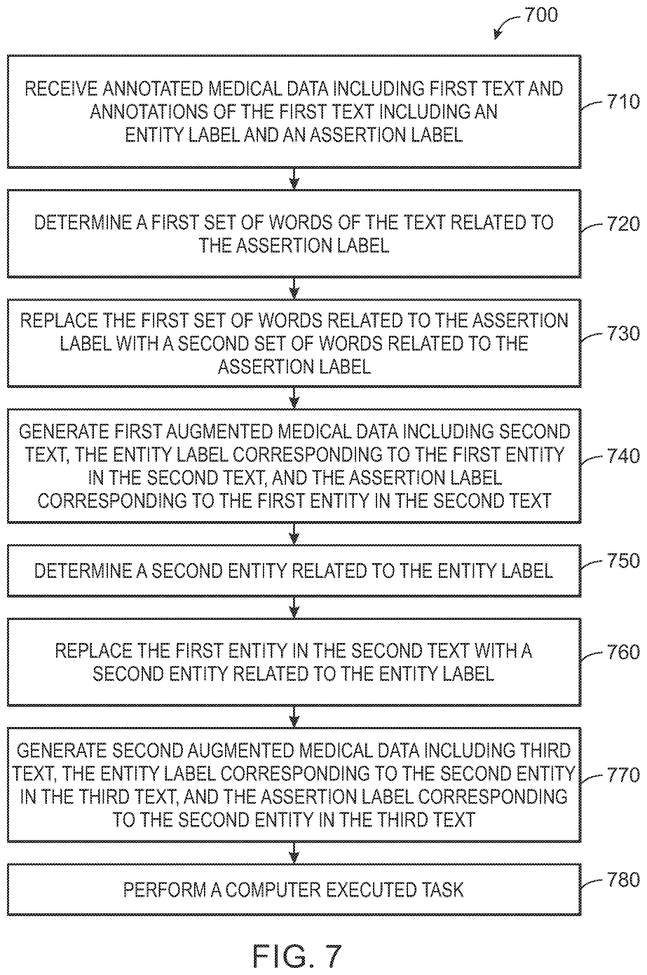

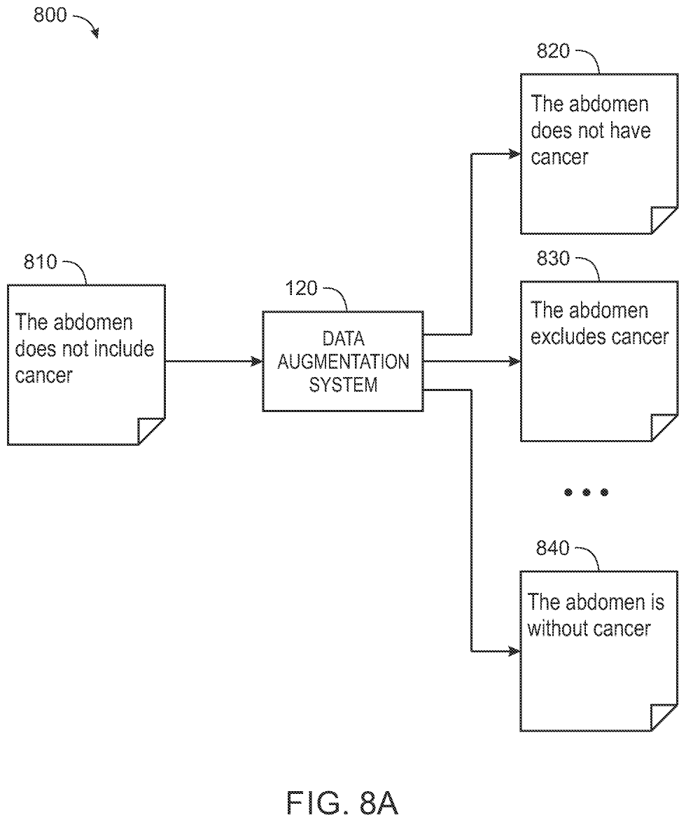

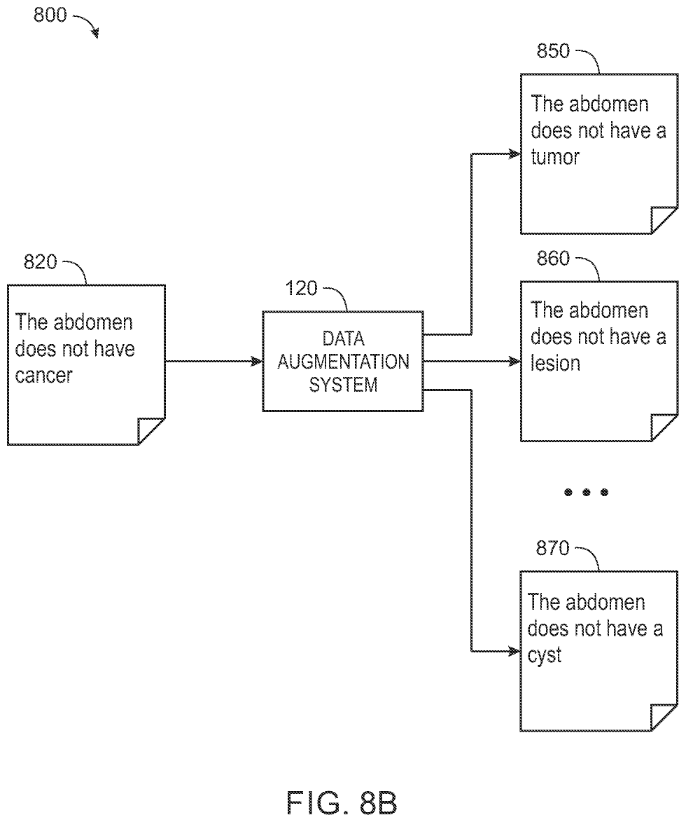

FIG. 7 is a flowchart of an example process for generating augmented medical data by replacing a set of words associated with an assertion label and by replacing an entity associated with an entity label. The data augmentation system 120 may be configured to perform the operations of the process 700. Alternatively, one or more other devices of FIG. 1 may be configured to perform one or more operations of the process 700. FIGS. 8A and 8B are diagrams of an example process for generating augmented medical data by replacing a set of words associated with an assertion label and by replacing an entity associated with an entity label.

As shown in FIG. 7, the process 700 may include receiving annotated medical data including first text, and annotations of the first text including an entity label corresponding to a first entity in the first text and an assertion label corresponding to the first entity in the text (operation 710), determining a first set of words of the text related to the assertion label (operation 720), replacing the first set of words related to the assertion label with a second set of words related to the assertion label (operation 730), and generating first augmented medical data including second text, the entity label corresponding to the first entity in the second text, and the assertion label corresponding to the first entity in the second text (operation 740).

For example, the data augmentation system 120 may perform similar operations as described above in connection with the process 300 of FIG. 3. As a particular example, and as shown in FIG. 8A, the data augmentation system 120 may receive annotated medical data 810, and generate augmented medical data 820 by replacing “does not include” with “does not have,” generate augmented medical data 830 by replacing “does not include” with “excludes,” and generate augmented medical data 840 by replacing “does not include” with “is without.”

As further shown in FIG. 7, the process 700 may include determining a second entity related to the entity label (operation 750), replacing the first entity in the second text with a second entity related to the entity label (operation 760), and generating second augmented medical data including third text, the entity label corresponding to the second entity in the third text, and the assertion label corresponding to the second entity in the third text (operation 770).

For example, the data augmentation system 120 may perform similar operations as described above in connection with the process 500 of FIG. 5. As a particular example, and as shown in FIG. 8B, the data augmentation system 120 may receive the augmented medical data 820, and generate augmented medical data 850 by replacing “cancer” with “tumor,” generate augmented medical data 860 by replacing “cancer” with “lesion,” and generate augmented medical data 870 by replacing “cancer” with “cyst.”

As further shown in FIG. 7, the process 700 may include performing a computer executed task using the augmented medical data (operation 780). For example, the computer executed task may include causing the clinical NLP model 160 to be trained, or training the clinical NLP model 160. As another example, the computer executed task may include causing the clinical NLP model 160 to be updated, or updating the clinical NLP model 160. As another example, the computer executed task may include transmitting the augmented medical data to another device. As another example, the computer executed task may include storing the augmented medical data in the augmented medical database 140. As another example, the computer executed task may include generating a user interface that displays the augmented medical data, and/or providing the user interface to another device.

In this way, the data augmentation system 120 may generate n pieces of augmented medical data by performing operations 710-740 in association with a piece of annotated medical data, and may generate m pieces of augmented medical data by performing operations 750-770 with respect to each piece of the n pieces of augmented medical data. Accordingly, the data augmentation system 120 may receive annotated medical data, generate an augmented medical data set including n variations of the annotated medical data by replacing words corresponding to an assertion label, and generate an augmented medical data set including m pieces of augmented medical data by replacing words corresponding to entities in each of the n variations of the medical data in the augmented medical data set. The data augmentation system 120 may store the augmented medical data set for subsequent training of the clinical NLP model 160. Accordingly, the data augmentation system 120 may quickly, efficiently, and accurately generate augmented medical data for training the clinical NLP model 160

Although FIG. 7 depicts the processes 700 as including a temporal order of operations 710-740 being performed before operations 750-770, in another embodiment, the operations 750-770 may be performed before the operations 710-740. In this case, the operations 710-740 may be performed based on augmented medical data generated by operations 750-770.

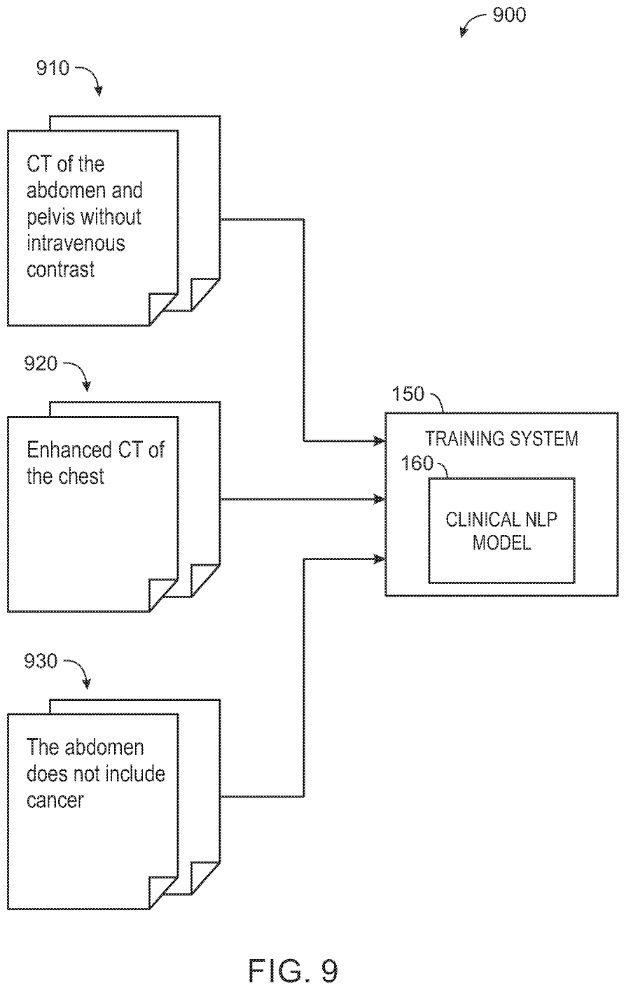

FIG. 9 is a diagram of an example process 900 for training a clinical NLP model. As shown in FIG. 9, the training system 150 may receive training data 910, training data 920, and training data 930. The training data 910 may be a training data set including annotated medical data and/or augmented medical data generated by replacing words associated with assertion labels. That is, the augmented medical data of the training data 910 may be generated based on operations associated with FIG. 3. The training data 920 may be a training data set including medical data and/or augmented medical data generated by replacing words associated with entity labels. That is, the augmented medical data of the training data 920 may be generated based on operations associated with FIG. 5. The training data 930 may be a training data set including medical data and/or augmented medical data generated by replacing words associated with assertion labels and entity labels. That is, the augmented medical data of the training data 930 may be generated based on operations associated with FIG. 7.

In this way, the training system 150 may train the clinical NLP model 160 using a greater amount of training data than as compared to using only annotated medical data that is annotated by a domain expert. Accordingly, the trained clinical NLP model 160 may be more accurate, more efficient, and/or less error-prone than as compared to models that are trained using only annotated medical data that is annotated by a domain expert.

Although embodiments herein describe generating augmented medical data for training a clinical NLP model 160, it should be understood that the embodiments herein are applicable to generating augmented data for training other types of models. For example, the embodiments herein are applicable to generating augmented data for training other types of models for performing other tasks such as image processing, decision-making, or the like.

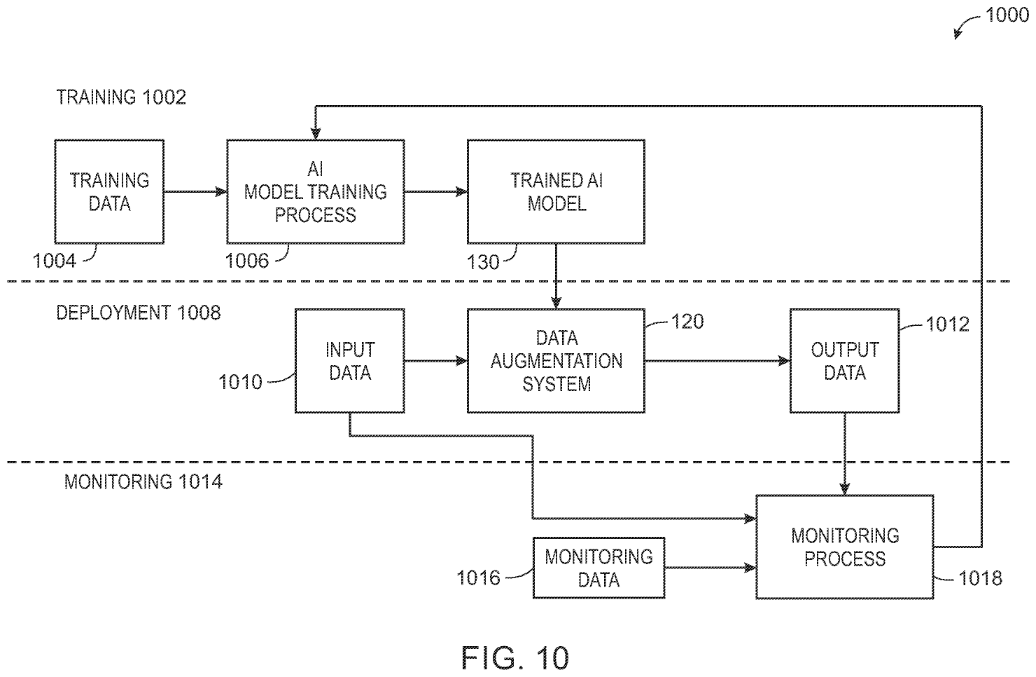

FIG. 10 is a diagram of an example process 1000 for training an AI model. The data augmentation system 120 may generate, store, train, and/or use the AI model 130. According to an embodiment, the data augmentation system 120 may include the AI model 130 and/or instructions associated with the AI model 130. For example, the data augmentation system 120 may include instructions for generating the AI model 130, training the AI model 130, using the AI model 130, etc. According to an embodiment, a system or device other than the data augmentation system 120 may be used to generate and/or train the AI model 130. For example, a system or device may include instructions for generating the AI model 130, and/or instructions for training the AI model 130. The system or device may provide a resulting trained AI model 130 to the data augmentation system 120 for use.

As shown in FIG. 10, according to an embodiment, the process 1000 may include a training phase 1002, a deployment phase 1008, and a monitoring phase 1014. In the training phase 1002, at operation 1006, the process 1000 may include receiving and processing training data 1004 to generate a trained AI model 130. The training data 1004 may be generated, received, or otherwise obtained from internal and/or external resources.

Generally, the AI model 130 may include a set of variables (e.g., nodes, neurons, filters, or the like) that are tuned (e.g., weighted, biased, or the like) to different values via the application of the training data 1004. According to an embodiment, the training process at operation 1006 may employ supervised, unsupervised, semi-supervised, and/or reinforcement learning processes to train the AI model 130. According to an embodiment, a portion of the training data 1004 may be withheld during training and/or used to validate the trained AI model 130.

For supervised learning processes, the training data 1004 may include labels or scores that may facilitate the training process by providing a ground truth. For example, the labels or scores may indicate an output of the AI model 130. Training may proceed by feeding a training dataset including the training data 1004 into the AI model 130. The AI model 130 may have variables set at initialized values (e.g., at random, based on Gaussian noise, based on pre-trained values, or the like). The AI model 130 may generate an output based on the training dataset being input to the AI model 130. The output may be compared with the corresponding label or score (e.g., the ground truth) indicating the known output, which may then be back-propagated through the AI model 130 to adjust the values of the variables. This process may be repeated for a plurality of samples at least until a determined loss or error is below a predefined threshold. According to an embodiment, some of the training data 1004 may be withheld and used to further validate or test the trained AI model 130.

For unsupervised learning processes, the training data 1004 may not include pre-assigned labels or scores to aid the learning process. Instead, unsupervised learning processes may include clustering, classification, or the like, to identify naturally occurring patterns in the training data 1004. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data 1004 with pre-assigned labels or scores and training data 1004 without pre-assigned labels or scores may be used to train the AI model 130.

When reinforcement learning is employed, an agent (e.g., an algorithm) may be trained to make a decision from the training data 1004 through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained AI model 130 may be stored and subsequently applied by the data augmentation system 120 during the deployment phase 1008. For example, during the deployment phase 1008, the trained AI model 130 executed by the data augmentation system 120 may receive input data 1010. During the deployment phase 1008, the trained AI model 130 may perform one or more operations as described in connection with FIGS. 3, 5, and 7.

After being deployed, the trained AI model 130 may be monitored during the monitoring phase 1014. For example, during the monitoring phase 1014, the AI model 130 may generate monitoring data 1016 that is used to monitor the trained AI model 130. The monitoring data 1016 may include data that identifies an output as determined by an operator. During process 1018, the monitoring data 1016 may be analyzed along with the predicted output data 1012 and input data 1010 to determine an accuracy of the trained AI model 130. According to an embodiment, based on the analysis, the process 800 may return to the training phase 1002, where at operation 1006 values of one or more variables of the model may be adjusted to improve the accuracy of the AI model 130.

The example process 1000 described above is provided merely as an example, and may include additional, fewer, different, or differently arranged aspects than depicted in FIG. 10.

FIG. 10 describes the training, deployment, and monitoring associated with a trained AI model 130 for determining an ECG interpretation result. According to an embodiment, one or more other trained AI model 130s may be applied. Each of the trained AI model 130s may include similar training, deployment, and/or monitoring phases as described above for the trained AI model 130 in FIG. 10, however the particular types of training data, input data, output data, and monitoring data may be different.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method comprising:
receiving annotated medical data including text and annotations of the text including an entity label corresponding to a first entity in the text and an assertion label corresponding to the first entity in the text;
replacing the first entity corresponding to the entity label with a second entity related to the entity label or replacing a first set of words related to the assertion label with a second set of words related to the assertion label;
generating augmented medical data based on replacing the first entity corresponding to the entity label with the second entity related to the entity label or replacing the first set of words related to the assertion label with the second set of words related to the assertion label; and
performing a computer executed task using the augmented medical data.

2. The method of claim 1, further comprising:
determining the first set of words using a regular expression.

3. The method of claim 1, further comprising:
determining the second set of words based on mapping information that maps the second set of words and the assertion label.

4. The method of claim 1, further comprising:
determining the second entity based on mapping information that maps the second entity with the entity label.

5. The method of claim 1, further comprising:
determining the second entity based on a frequency of the second entity in stored medical data.

6. The method of claim 1, further comprising:
determining the second entity comprises using stored medical data that includes the entity label and the assertion label.

7. The method of claim 1, wherein performing the computer executed task using the augmented medical data comprises:
training a clinical natural language processing (NLP) model using the augmented medical data.

8. A method comprising:
receiving annotated medical data including first text and annotations of the first text including an entity label corresponding to an entity in the first text and an assertion label corresponding to the entity in the first text;
determining a first set of words of the first text related to the assertion label;
replacing the first set of words related to the assertion label with a second set of words related to the assertion label;
generating augmented medical data including second text, the entity label corresponding to the entity in the second text, and the assertion label corresponding to the entity in the second text, based on replacing the first set of words related to the assertion label with the second set of words related to the assertion label; and
performing a computer executed task using the augmented medical data.

9. The method of claim 8, wherein the entity is a first entity, and wherein the method further comprises:
determining a second entity related to the entity label;
replacing the first entity in the second text with the second entity related to the entity label; and
generating second augmented medical data including third text and the entity label corresponding to the second entity in the third text, based on replacing the first entity in the second text with the second entity related to the entity label.

10. The method of claim 8, wherein determining the first set of words comprises determining the first set of words using a regular expression.

11. The method of claim 8, further comprising:
determining the second set of words based on mapping information that maps the second set of words and the assertion label.

12. The method of claim 9, wherein determining the second entity comprises determining the second entity based on mapping information that maps the second entity with the entity label.

13. The method of claim 9, wherein determining the second entity comprises determining the second entity based on a frequency of the second entity in stored medical data.

14. The method of claim 9, wherein determining the second entity comprises determining the second entity using stored medical data that includes the entity label and the assertion label.

15. A method comprising:
receiving annotated medical data including first text and annotations of the first text including an entity label corresponding to a first entity in the first text and an assertion label corresponding to the first entity in the first text;
determining a second entity related to the entity label;
replacing the first entity in the first text with the second entity related to the entity label;
generating augmented medical data including second text, the entity label corresponding to the second entity in the second text, and the assertion label corresponding to the second entity in the second text, based on replacing the first entity in the second text with the second entity related to the entity label; and
performing a computer executed task using the augmented medical data.

16. The method of claim 15, further comprising:
determining a first set of words of the first text related to the assertion label; and
replacing the first set of words related to the assertion label with a second set of words related to the assertion label.

17. The method of claim 15, wherein determining the second entity comprises determining the second entity based on mapping information that maps the second entity with the entity label.

18. The method of claim 15, wherein determining the second entity comprises determining the second entity based on a frequency of the second entity in stored medical data.

19. The method of claim 15, wherein determining the second entity comprises determining the second entity using stored medical data that includes the entity label and the assertion label.

20. The method of claim 15, wherein performing the computer executed task using the augmented medical data comprises:
training a clinical natural language processing (NLP) model using the augmented medical data.
